(19) United States
(12) Patent Application Publication
(10) Pub. No.: US 2005/0257793 A1
(43) Pub. Date: Nov. 24, 2005

(54) Nasal inhalation device

(76) Inventor: Tsuyoshi Tatsumoto, Tokyo (JP)

Correspondence Address: MORRISON & FOERSTER LLP, 755 PAGE MILL RD, PALO ALTO, CA, 94304-1018, US

(21) Appl. No.: 10/849,483

(22) Filed: May 18, 2004

(51) Int. Cl. A61G 10/00; A61M 16/00

(52) U.S. Cl. 128/207.18; 128/206.11

(57) ABSTRACT

A nasal inhalation device includes a body and at least one cover. The body includes a connective portion and at least one dilating portion provided on one end of the connective portion, the dilating portion having a cavity. The cover is connected to the dilating portion, and capable of being assembled with the dilating portion to form a receiving unit. The receiving unit can be inserted into a nostril to provide nasal absorption or inhalation of substances contained in the receiving unit for a user.

### FIELD OF THE INVENTION

The present invention relates to nasal inhalation devices, and more particularly, to a nasal inhalation device allowing direct nasal absorption or inhalation of substances for a user.

### BACKGROUND OF THE INVENTION

Along with improved living quality in present days, different environmental fragrances or essential oils are frequently used to diffuse aromatic particles into air to produce pleasing smells or odors in the environments where people are usually situated. However, each individual person may have different senses of smell and favor different kinds of odors, such that the dispersed aromatic particles might not be acceptable for each individual. Moreover, for users requiring direct nasal absorption or inhalation of specific substances, it is thus not appropriate to disperse such specific substances into the environments. Therefore, in recent years, various nasal inhalation devices are provided to individual users for nasal inhalation of different substances including fragrances, drugs (such as painkillers) or other nasal inhaled or absorptive substances. The users can absorb or inhale different fragrances, medicaments or other substances through nasal cavities depending on personal requirements without affecting other people's sense of smell.

U.S. Pat. No. 6,422,234 discloses a nasal dispenser 1′ as shown in FIG. 12. This nasal dispenser 1′ comprises a container 2′ mounted in a body 3′ of the nasal dispenser 1′, a piston 15′ housed in the body 3′, and a polyethylene tube 11′ for connecting the container 2′ to the piston 15′. The piston 15′ can be shifted toward or away from an inhalation orifice 20′ of the nasal dispenser 1′ by adjusting a spring 23′. Thereby medicaments or other substances contained in the contained 2′ can be inhaled through the inhalation orifice 20′ by a user.

However, the above nasal dispenser 1′ must be held by the user's hand to perform the nasal absorption or inhalation. It is thus not conveniently used for a user who is engaged in job, class or meeting and for those who are disabled. Further, the nasal dispenser 1′ is usually sized relatively larger for easy holding, making the nasal dispenser 1′ not highly portable. Additionally, the nasal dispenser 1′ is inherent with significant drawbacks in practical use, for example, a trouble of filling the substances for absorption or inhalation in the container 2′.

Therefore, in order for the users to easily absorb or inhale the substances such as medicaments or fragrances, the applicant of the present invention filed a patent application directed to a nasal insufflation device 10 to be inserted into nostrils 3 as shown in FIGS. 13 and 14, which has been granted Japanese Patent No. 2001-009034.

Referring to FIGS. 13 and 14, the nasal insufflation device 10 comprises a pair of aromatic bodies 101 that can be inserted into the nostrils 3 respectively, and an elastic material 103 for connecting the pair of aromatic bodies 101 together. The elastic material 103 facilitates the insertion of the aromatic bodies 101 into the nostrils 3. Each of the aromatic bodies 101 comprises an upper container 101a and a lower container 101b that are coupled to each other. A plurality of micropores 101c are provided on surfaces of the upper container 101a and the lower container 101b, and carrier particles (not shown) of fragrances or medicaments are packed in the aromatic bodies 101. This allows molecules of the fragrances or medicaments to be released from the aromatic bodies 101 through the micropores 101c and inhaled by the nostrils 3 during normal respiration.

The above nasal insufflation device 10 desirably operates without being held by the user's hand. However, as shown in FIG. 13, the aromatic bodies 101 of the nasal insufflation device 10 cannot be elastically bent along with the elastic material 103, making the aromatic bodies 101 not able to be adjusted in position inside the nostrils 3 according to different structure of nasal cavities 5 and not providing good support.

Further referring to FIG. 14, when the aromatic bodies 101 of the nasal insufflation device 10 is inserted into the nostrils 3 and located at opposite sides of a nasal septum 7, there is no clamping structure to secure the aromatic bodies 101 to the nasal septum 7 such that no support is provided at this position. As a result, the nasal insufflation device 10 cannot be firmly held in the nostrils 3, but may easily drop and become dirtied or damaged. Therefore, it is difficult to alter or maintain the inserting position of the nasal insufflation device 10 in the nostrils 3. Moreover, connection between the aromatic bodies 101 and the elastic material 103 sets a restriction on adjusting the location of the aromatic bodies 101 in the nostrils 3.

Furthermore, the upper container 101a and the lower container 101b of the nasal insufflation device 10 are separate and need to be assembled together by both hands while difficult to be disassembled. Such an operation is not easy for normal people, especially hard for children, the elders or those whose hands are disabled, such that the nasal insufflation device 10 is practically not convenient to use. Particularly it would be a trouble to repeatedly disassemble and assemble the upper container 101a and the lower container 101b for adding or replacing the substances contained in the aromatic bodies 101.

Therefore, the problem to be solved here is to provide a nasal inhalation device, which can eliminate the above prior-art drawbacks such as relatively large size, poor supportability, and restriction on inserting positions, which result in inconvenience for use, high fabrication cost, difficulty in carrying, hard adjustment and dirtiness or damage to conventional nasal inhalation devices.

### SUMMARY OF THE INVENTION

A primary objective of the present invention is to provide a nasal inhalation device easy to use.

Another objective of the present invention is to provide a nasal inhalation device whose inserting position in nostrils can be adjusted and secured depending on user's requirement.

Still another objective of the present invention is to provide a nasal inhalation device to be inserted into nostrils without using the hands.

A further objective of the present invention is to provide a nasal inhalation device that is portable and convenient to carry.

A further objective of the present invention is to provide a nasal inhalation device easy to be fabricated.

In accordance with the above and other objectives, the present invention proposes a nasal inhalation device comprising a body and at least one cover.

The body comprises a connective portion and at least one dilating portion provided at one end of the connective portion, the dilating portion having a cavity. The dilating portion is formed with a first positioning portion, and the cover is formed with a second positioning portion corresponding to the first positioning portion, such that the dilating portion and the cover can be assembled by means of the first and second positioning portions to form a receiving unit that is to be inserted into a nostril and properly positioned. A plurality of air-permeable portions is respectively provided on surfaces of the dilating portion and the cover, allowing the user to absorb or inhale the substances contained in the receiving unit through the air-permeable portions. Further, the connective portion is made of a flexible material, which can be adjusted to place and support the receiving unit in a proper position into the nostril depending on the use's requirement.

The dilating portion, the connective portion and the cover of the nasal inhalation device in the present invention may be integrally fabricated, and the mold for forming the air-permeable portions is more easily prepared as compared to the prior art, thereby simplifying the fabrication processes and reducing the fabrication cost of the nasal inhalation device.

The nasal inhalation device proposed in the present invention can desirably avoid the prior-art drawbacks, which beneficially has significant advantages such as relatively smaller size, good supportability and ease to adjust, allowing the user to insert the nasal inhalation device into one or two nostrils without using both hands. Therefore, the nasal inhalation device in the present invention is convenient to use and highly portable, and would not easily drop out from the nostrils to get dirtied or damaged, as well as has the simplified fabrication processes and reduced fabrication cost.

### BRIEF DESCRIPTION OF THE DRAWINGS

The present invention can be more fully understood by reading the following detailed description of the preferred embodiments, with reference made to the accompanying drawings, wherein:

FIG. 1 is a schematic diagram showing a nasal inhalation device according to a first preferred embodiment of the present invention;

FIG. 2 is a top view of the nasal inhalation device according to the first preferred embodiment of the present invention;

FIG. 3 is a bottom view of the nasal inhalation device according to the first preferred embodiment of the present invention;

FIG. 4 is a cross-sectional view of the nasal inhalation device of FIG. 2 along line A-A;

FIG. 5 is an operational schematic diagram of a body of the nasal inhalation device of FIG. 1 placed in nasal cavities;

FIG. 6 is another operational schematic diagram of the body of the nasal inhalation device of FIG. 1 placed in the nasal cavities and not in contact with walls of the nasal cavities;

FIG. 7 is an operational schematic diagram of the body of the nasal inhalation device of FIG. 1 placed in nostrils and in contact with a nasal septum;

FIG. 8 is another operational schematic diagram of the body of the nasal inhalation device of FIG. 1 placed in the nostrils and not in contact with the nasal septum;

FIG. 9 is a cross-sectional view of a nasal inhalation device according to a second preferred embodiment of the present invention;

FIGS. 10A and 10B are operational schematic diagrams of the nasal inhalation device according to the second preferred embodiment of the present invention;

FIG. 11 is another operational schematic diagram of the nasal inhalation device according to the second preferred embodiment of the present invention;

FIG. 12 (PRIOR ART) is a schematic diagram of a conventional nasal dispenser;

FIG. 13 (PRIOR ART) is an operational schematic diagram of a conventional nasal insufflation device with its aromatic bodies placed in nasal cavities; and

FIG. 14 (PRIOR ART) is another operational schematic diagram of the conventional nasal insufflation device with its aromatic bodies located at a nasal septum.

### DETAILED DESCRIPTION OF THE PREFERRED EMBODIMENTS

### First Preferred Embodiment

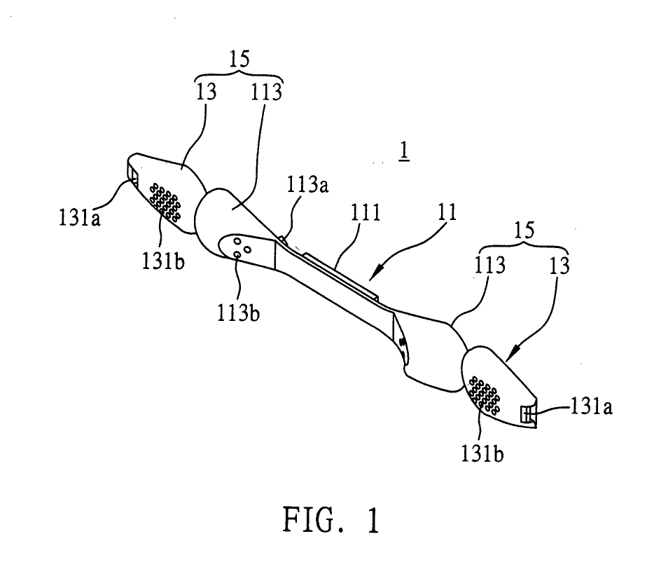

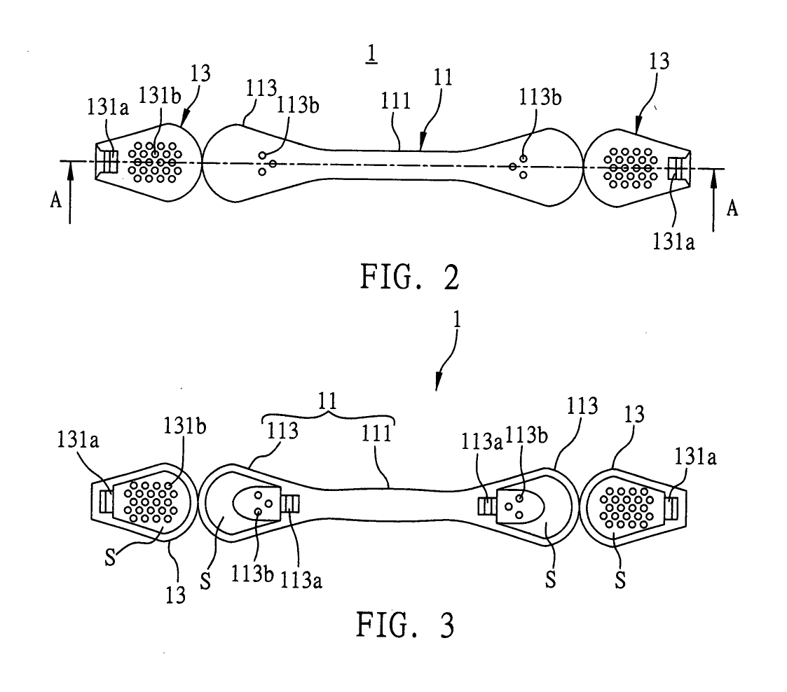

FIGS. 1 to 8 show a nasal inhalation device 1 according to the first preferred embodiment of the present invention. This nasal inhalation device 1 comprises a spindle-shaped body 11 and two covers 13 connected to the body 11. In this embodiment, the nasal inhalation device is described for use in nostrils of the nose, and thus only part of structure of the nose related to operation of the nasal inhalation device is shown here.

Referring to FIGS. 1 to 3, the body 11 comprises a connective portion 111 and two dilating portions 113 respectively connected to two ends of the connective portion 111, wherein the dilating portions 113 are each formed with a cavity S.

The two ends of the connective portion 111 and the dilating portions 113 may be integrally formed. The connective portion 111 is made of a flexible material having elastic resilience and preferably having a straight structure. The connective portion 111 can be deformed by a force applied from a user depending on personal requirement. When the applied force is removed, the connective portion 111 would remain in the deformed shape, and can be changed to another shape by exerting another force on the connective portion 111.

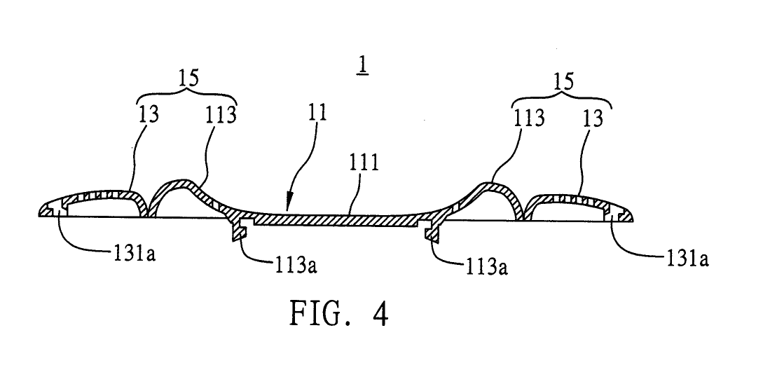

The dilating portions 113 are connected to the covers 13 respectively and can be integrally formed with the covers 13. The dilating portion 113 is provided with a first positioning portion 113a corresponding to a second positioning portion 131a of the cover 13. Similarly, the cover 13 is also provided with a cavity S corresponding to the cavity S of the dilating portion 113. As shown in FIG. 4, the first positioning portion 113a may be shaped as a protrusion having a hook, and the second positioning portion 131a may be correspondingly shaped as a recess to be engaged with the protrusion, such that the dilating portion 113 and the cover 13 can be easily closed with each other to form a receiving unit 15 or can be easily opened from each other by coupling or uncoupling the first and second positioning portions 113a, 131a.

By virtue of the above flexible characteristic of the connective portion 111, the receiving unit 15 connected to the connective portion 111 can be adjusted in its location placed in a nostril (not shown) depending on the user's requirement and stably supported in position.

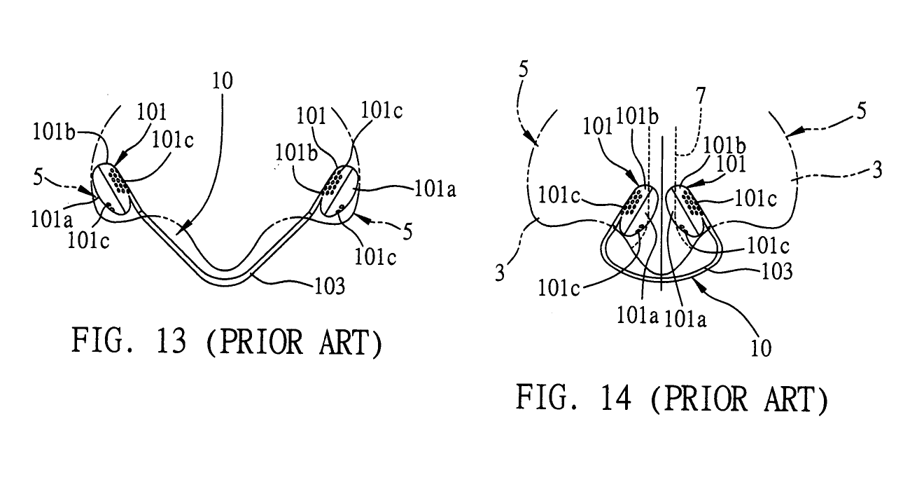

The integrally-formed connective portion 111 and dilating portions 113 desirably solve the prior-art problem of a restriction on position adjustment for the aromatic bodies 101 and the elastic material 103 in FIGS. 13 and 14. Also, the size of the nasal inhalation device 1 can be reduced relative to the conventional device. The nasal inhalation device 1 having a reduced size is easier and more convenient to be used by the user to place the nasal inhalation device at a desirable position without affecting the user's daily life.

The shape and thickness of the connective portion 111 are not limited to those shown in this embodiment, which may be flexibly arranged as long as the receiving units 15 can be placed at a proper position in the nostrils without impeding the user's breath. The flexible material for fabricating the connective portion 111 is known in the art and not to be further detailed.

Further referring to FIGS. 1 to 4, since the dilating portion 113 and the cover 13 are connected together, the first positioning portion 113a and the second positioning portion 131a can be engaged with or separated from each other simply by using a single hand, not requiring both hands, to close or open the receiving unit 15 composed of a pair of the dilating portion 113 and the cover 13. As such, substances for nasal absorption or inhalation can be easily added or replaced in the body 11. Therefore, the nasal inhalation device 1 in the present invention is more convenient and easy to use as compared to the conventional nasal inhalation device.

Apart from the engagement connection between the first positioning portion 113a and the second positioning portion 131a for closing/opening the dilating portion 113 and the cover 13 relative to each other, other connection methods are also suitable such as locking, scarf, concave/convex fitting, hooking, clinching, adsorption and so on. In other words, the first positioning portion 113a and the second positioning portion 131a can be selected from blocks, recesses, locking pawls, closed grooves, guide shoes, snap rings, piercing holes, lugs, springs, elastic fragments, magnets and other equivalent elements, which are detachably coupled to each other and allow the receiving unit 15 to be easily opened or closed.

A plurality of porous air-permeable portions 113b and 131b are respectively formed on the dilating portion 113 and the cover 13, wherein the air-permeable portions 113b on the dilating portion 113 are more densely arranged than the air-permeable portions 131b on the cover 13. It should be understood that the arrangement, shape, size and number of the air-permeable portions 113b and 131b are not limited to those shown in this embodiment but can be flexibly adjusted. For example, in order to shorten time and reduce cost for mold fabrication, the air-permeable portions 113b and 131b may be made as rectangular holes or other structures that allow molecules of fragrances or medicaments to easily go through the air-permeable portions 113b and 131b and inhaled by the nose.

I've looked them all up and have written our client matter numbers on them. Moreover, the substances such as carrier particles, ointments and solidified substances of fragrances or medicaments can be stored in the receiving units 15 each composed of a pair of the dilating portion 113 and the cover 13, such that odor molecules of the fragrances or medicaments can be absorbed or inhaled through the body 11 by the user. The nasal inhalation device 1 in the present invention is preferably made of antiseptic materials to prevent the user from discomfort or symptom such as inflammation.

Since the nasal inhalation device 1 can be fabricated with the integrally-formed dilating portions 113 and covers 13 and the integrally-formed connective portion 111 and dilating portions 113, as discussed above, the nasal device 1 allows the use to easily open and close the receiving units 15, and also reduce the time and cost for mold fabrication as compared to the prior art, thereby making the nasal inhalation device 1 simpler and more time- and cost-effective to fabricate.

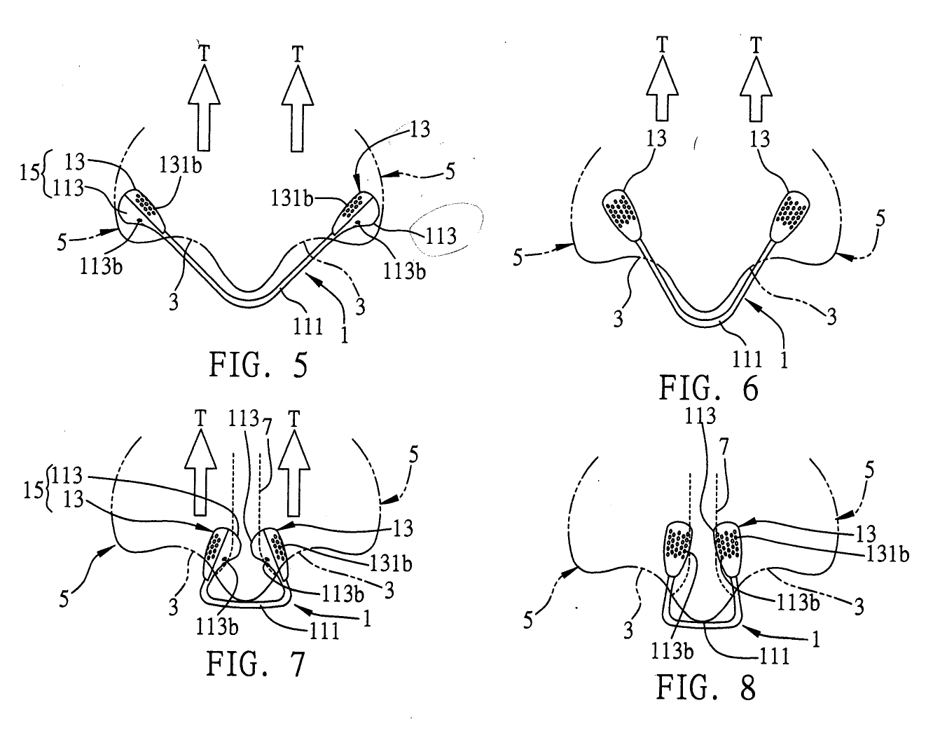

Referring to FIG. 5, when the receiving units 15 of the nasal inhalation device 1 are respectively inserted into nasal cavities 5 through the nostrils 3, in respect of each one of the receiving units 15, a surface of the dilating portion 113 of the body 11 abuts against a surface of the nasal cavity 5, and the cover 13 faces away from the surface of the nasal cavity 5 (i.e. facing toward a pathway of odor inhalation indicated by arrows T), such that the user can absorb or inhale the odor molecules of fragrances or medicaments contained in the body 11 through the air-permeable portions 131b on the surface of the cover 13. By the above arrangement, the user absorbs or inhales more odor molecules with the surface of the cover 13 having the air-permeable portions 131b facing toward the pathway of odor inhalation. Contrarily, if the user prefers to absorb or inhale less odor molecules, it is to make the dilating portion 113 face away from the surface of the nasal cavity 5 and the surface of the cover 13 abut against the surface of the nasal cavity 5, as shown in FIG. 6.

Since the dilating portions 113 of the body 11 and the covers 13 are designed according to the nasal cavities 5 of the nostrils 3, the surfaces of the dilating portions 113 and the covers 13 can either be closely in contact with the surfaces of the corresponding nasal cavities 5, such that the receiving units 15 can be retained in the nasal cavities 5 of the nostrils 3 even when changing the position thereof in the nasal cavities 5. Therefore, the nasal inhalation device 1 in the present invention can solve the prior-art problem that the conventional nasal inhalation device may drop out from the nostrils, and also allows the user to comfortably use the nasal inhalation device 1 for a long period of time.

Referring to FIG. 7, when the receiving units 15 of the nasal inhalation device 1 are attached to surfaces of the nasal septum 7 through the nostrils 3, the dilating portions 113 of the body 11 can be adapted to adequately clamp the surfaces of the nasal septum 7, and the covers 13 face away from the surfaces of the nasal septum 7 (i.e. facing toward a pathway of odor inhalation indicated by arrows T), allowing the user to absorb or inhale more odor molecules of the fragrances or medicaments through the air-permeable portions 131b on the surface of the covers 13. Contrarily, if the user prefers to absorb or inhale less odor molecules, it is to make the dilating portions 113 face away from the surfaces of the nasal septum 7 and the covers 13 appropriately clamp the surfaces of the nasal septum 7.

Thus, the desirable effect of nasal absorption or inhalation can be achieved using the nasal inhalation device 1. As the dilating portions 113 and the covers 13 are respectively adapted to adequately clamp the nasal septum 7, the prior-art problem that the conventional nasal inhalation device may drop out from the nostrils can be solved, and the nasal inhalation device 1 in the present invention can be comfortably used by the user for a long period of time. Further referring to FIGS. 5 and 7, the receiving units 15 of the nasal inhalation device 1 in the present invention can be placed in different manners and positions in the nasal cavities 5 through the nostrils 3 depending on the user's requirement, such that the prior-art drawback that the conventional nasal inhalation device must be held by both hands is eliminated, and the user can comfortably use the nasal inhalation device 1 for a long period of time.

Referring to FIGS. 6 and 8, when the substances (not shown) contained in the receiving units 15 should not contact the nasal mucus in the nasal cavities 5, the flexible connective portion 111 allows the position of the receiving units 15 in the nasal cavities 5 to be adjusted making the air-permeable portion 113b of the dilating portions 113 of the body 11 and the air-permeable portion 131b of the covers 13 not in direct contact with the surfaces of the nasal cavities 5 and nasal septum 7. As a result, the surfaces of the dilating portions 113 and the covers 13 not having the air-permeable portion 113b, 131b abut against the surfaces of the nasal cavities 5 and the nasal septum 7. As discussed above, the connective portion 111 is deformed upon a force and remains being deformed even when the force is released, thereby making the receiving units 15 firmly held in the adjusted position in the nasal cavities 5 through the use of the connective portion 111. Therefore, nasal absorption or inhalation of any desirable substances can be performed by inserting the receiving units 15 of the nasal inhalation device 1 into the nostrils 3 and adjusting the position of the receiving units 15 in the nasal cavities 5 using the flexible connective portion 111.

### Second Preferred Embodiment

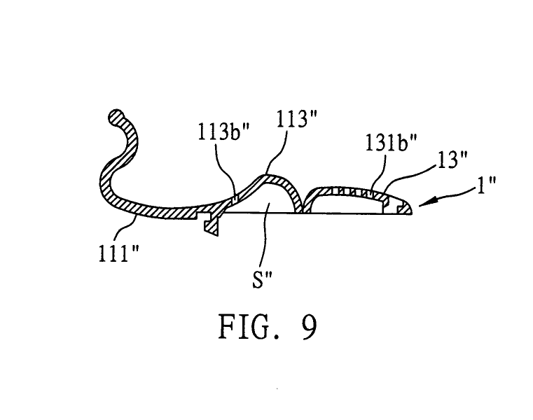

FIGS. 9 to 11 show a nasal inhalation device 1″ according to a second preferred embodiment of the present invention. Referring to FIG. 9, the nasal inhalation device 1″ comprises a spindle-shaped body 11″ and a cover 13″ connected to the body 11″. The function and structure of the nasal inhalation device 1″ in this embodiment are similar to those of the nasal inhalation device 1 in the first embodiment. Thus, only differences in structure and function between the two embodiments are described here.

The nasal inhalation device 1″ in this embodiment differs from the nasal inhalation device 1 of the first embodiment in that, the body 11″ comprises a connective portion 111″ and a dilating portion 113″ connected to only one end of the connective portion 111″, wherein the dilating portion 113″ is formed with a cavity S″, and the connective portion 111″ is a bent flexible structure.

Referring to FIGS. 10A and 10B, the connective portion 111″ is fabricated according to ergonomics and can be adjusted according to the shape of the user's nose. The dilating portion 113″ and the cover 13″ are coupled together to form a receiving unit 15″. The user can adjust the relative positions of the receiving unit 15″ and the connective portion 111″ depending on the personal requirement to comfortably wear the nasal inhalation device 1″ on the nose. As shown in the drawings, for example, the nasal inhalation device 1″ is clipped on a side of the nostril 3.

Similarly to the foregoing embodiment, it allows to change the position of the nasal inhalation device 1″ on the nose while securing the nasal inhalation device 1″ in position to provide desirable effect of nasal absorption or inhalation. For example, air-permeable portions 113b″ on the dilating portion 113″ and/or air-permeable portions 131b″ on the cover 13″ are optionally positioned to prevent contact with the nasal mucus (not shown). It should be understood that the position of the receiving unit 15″ is not limited to those shown in this embodiment but can be flexibly adjusted.

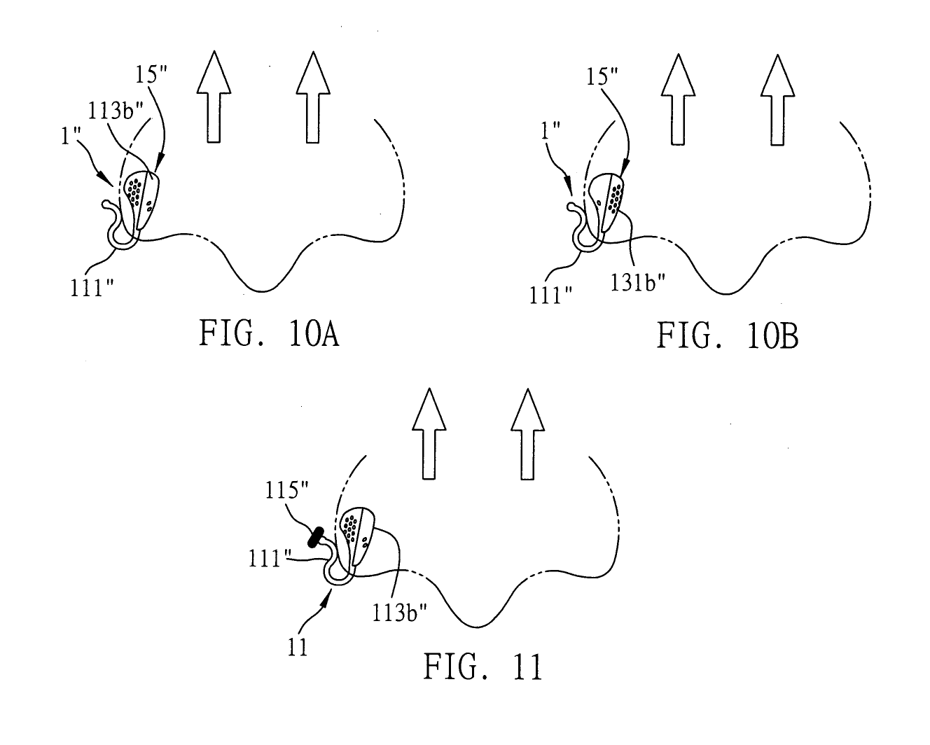

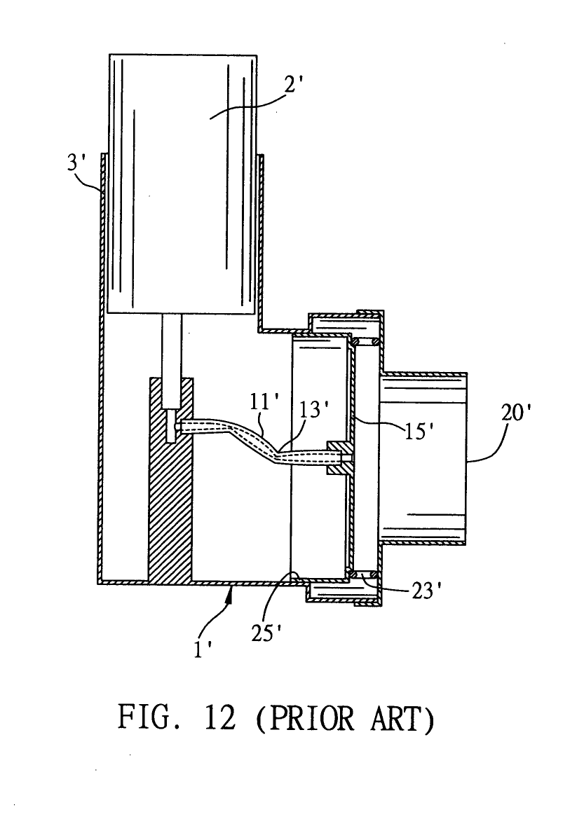

Referring to FIG. 11, a decorative portion 115″ may be formed at the end of the connective portion 111″ of the body 11″ not connected with the dilating portion 113″. This decorative portion 115″ can have a different shape from that of the connective portion 111″, and is adapted to aid or decorate the adjustment function of the connective portion 111″. The decorative portion 115″ and the connective portion 111″ can be integrally fabricated, or separately formed to be capable of being disassembled from each other, and their structures are not limited to those shown in this embodiment.

Therefore, the nasal inhalation device proposed in the present invention has significant benefits such as ease in use and fabrication, being firmly placed into the nostrils without being held by both hands, being convenient to carry or portable, being adjusted and held in position depending on the user's requirement, being comfortable to use, good appearance, providing aided or decorative adjustment function, and reducing the fabrication cost.

It should be understood that, the substances stored in the nasal inhalation device in the present invention are not limited to fragrances or medicaments described in the above embodiments, but can be any substances for nasal absorption or inhalation preferred by the user. Further, the size and number of the air-permeable portions are flexibly adjusted not limited to those shown in the above embodiments.

Accordingly, the dilating portion and the cover of the nasal inhalation device proposed in the present invention are connected to each other, and the connective portion of the body and the dilating portion are integrally formed to be placed into the nostrils. Therefore, the prior-drawbacks of requiring both hands to hold the conventional nasal inhalation device and difficulty in adding or replacing substances in the conventional nasal inhalation device can be eliminated in the present invention, such that the elders, children or those who are disabled can easily use the nasal inhalation device proposed in the present invention.

The position of the receiving unit in the nasal cavity can be adjusted using the connective portion of the nasal inhalation device proposed in the present invention depending on the user's requirement, and can be stably supported in the nasal cavity. For example, the receiving unit is optionally inserted into one or two nostrils, and the position of the receiving unit can be altered to provide desirable effect of nasal absorption or inhalation.

The nasal inhalation device proposed in the present invention can be placed and firmly held in position in the nostrils, thereby eliminate the prior-art drawback of requiring both hands to hold the conventional nasal inhalation device, such that the user can use the nasal inhalation device in the present invention at any time or location where the hands are occupied such as during class, meeting or work and for a long period of time without affecting the daily life.

Since the nasal inhalation device in the present invention is used not requiring both hands, the size thereof can be made much smaller than that of the conventional nasal inhalation device operated by both hands. Further, the flexible connective portion of the nasal inhalation device in the present invention can be deformed to reduce the volume of the nasal inhalation device that is thus conveniently carried by the user.

The connective portion, the dilating portion and the cover of the nasal inhalation device in the present invention may be integrally formed, such that the time and cost for mold fabrication are reduced thereby making the nasal inhalation device simple, time-effective and cost-effective to fabricate.

Therefore, the nasal inhalation device proposed in the present invention desirably avoids the prior-art drawbacks such as inconvenience in use, high fabrication cost, difficulty in carrying, hard adjustment, contamination or damage to the conventional nasal inhalation device if it drops out from the nostrils. The nasal inhalation device in the present invention can be easily used by different users and adjusted in its location in the nostrils while firmly supported in position depending one the user's requirement. The nasal inhalation device in the present invention does not require both hands to operate, and is portable and conveniently used anytime as well as economical due to its simple fabrication.

The invention has been described using exemplary preferred embodiments. However, it is to be understood that the scope of the invention is not limited to the disclosed embodiments. On the contrary, it is intended to cover various modifications and similar arrangements. The scope of the claims, therefore, should be accorded the broadest interpretation so as to encompass all such modifications and similar arrangements.

1: A nasal inhalation device comprising:
a spindle-shaped body comprising a connective portion and at least one dilating portion provided on one end of the connective portion, the dilating portion having a cavity; and
at least one cover connected to the dilating portion of the body, allowing the cover to be assembled with the dilating portion to form at least one receiving unit that is to be placed into a nostril.

2: The nasal inhalation device of claim 1, wherein the dilating portion is formed with a first positioning portion, and the cover is formed with a second positioning portion corresponding to the first positioning portion, so as to detachably assemble the dilating portion and the cover by means of the first and second positioning portions.

3: The nasal inhalation device of claim 2, wherein the first positioning portion is an element selected form the group consisting of block, recess, locking pawl, closed groove, guide shoe, snap ring, piercing hole, lug, spring, elastic fragment and magnet.

4: The nasal inhalation device of claim 2, wherein the second positioning portion is an element selected form the group consisting of block, recess, locking pawl, closed groove, guide shoe, snap ring, piercing hole, lug, spring, elastic fragment and magnet.

5: The nasal inhalation device of claim 1, wherein a plurality of air-permeable portions are provided on the dilating portion.

6: The nasal inhalation device of claim 1, wherein a plurality of air-permeable portions are provided on the cover.

7: The nasal inhalation device of claim 1, wherein the body contains substances for absorption or inhalation that are selected from the group consisting of carrier particles, ointments and solidified substances.

8: The nasal inhalation device of claim 1, wherein the dilating portion and the cover are integrally formed.

9: The nasal inhalation device of claim 1, wherein the connective portion, the dilating portion and the cover are integrally formed.

10: The nasal inhalation device of claim 1, wherein the connective portion is made of a flexible material.

11: The nasal inhalation device of claim 10, wherein the connective portion is a straight flexible structure.

12: The nasal inhalation device of claim 10, wherein the connective portion is a bent flexible structure.

13: The nasal inhalation device of claim 12, wherein a decorative portion is provided on the other end of the connective portion.
